(19) United States
(12) Patent Application Publication
(10) Pub. No.: US 2008/0131504 A1
(43) Pub. Date: Jun. 5, 2008

(54) Short Term Slow Release Drug Delivery System

(75) Inventors: Neill B. Walsdorf, Sr., San Antonio, TX (US); Jon C. Taylor, San Antonio, TX (US); Feng Zhang, Austin, TX (US); Francis K. Sherwood, Austin, TX (US); John J. Koleng, Austin, TX (US)

Correspondence Address: FULBRIGHT & JAWORSKI, LLP, 1301 MCKINNEY, SUITE 5100, HOUSTON, TX, 77010-3095, US

(73) Assignee: Mission Pharmacal Co., San Antonio, TX (US)

(21) Appl. No.: 11/565,846

(22) Filed: Dec. 1, 2006

(51) Int. Cl. A61K 9/20; A61K 33/00; A61K 33/32

(52) U.S. Cl. 424/464; 424/722; 424/639

(57) ABSTRACT

The present invention is directed to a novel short term slow release drug delivery system, preferably for solid oral dosage forms of water-soluble, alkaline salts of alkali metals and alkaline earth metals comprising polyvinylpyrrolidone and CPAA and preferably a wax component.

### TECHNICAL FIELD

The present invention relates to a novel short term slow release drug delivery system for solid oral dosage forms of water-soluble, alkaline salts of alkali metals and alkaline earth metals comprising polyvinylpyrrolidone and a cross-linked polyacrylic acid and preferably a wax component.

### BACKGROUND OF THE INVENTION

The alkaline salts of potassium are each useful in the treatment and prevention of hypokalemia, thiazide induced hypokalemia, as well as uric acid and calcium oxalate kidney stones. Other alkali and alkaline earth metal salts have various therapeutic uses.

Solid oral dosage forms of water-soluble alkali metal and alkaline earth metal salts are preferably provided in large doses (typically greater than 1 g per dosage unit). Such large dosage units of these active pharmaceutical ingredients present problems. It is difficult to produce a suitable pharmaceutical product using these salts because they are all somewhat hygroscopic and because the ions are both irritating and somewhat erosive to the gastric mucosa, each must be given in a slow release form. Historically, wax matrix tablets or microencapsulated products have been used to avoid poor patient acceptance and poor dosage compliance.

It is very difficult with the above manufacturing technicalities to produce a high dosage content product. In general, patients are required to take multiple tablets, or multiple dosage regimes to accomplish good steady state levels of these salts.

The systems disclosed here provide a well tolerated dosage with as much as 50% greater active dosage per tablet, using a very efficient combination of retardants requiring about one-half the total volume of retarding excipient materials normally needed to achieve a desirable release rate.

An example of a preferred active pharmaceutical ingredient is potassium magnesium citrate (KMC). Attempts to prepare 10 mEq sustained release KMC tablets using a simple matrix based on hydrophilic and hydrophobic polymers as drug release retardants were not successful. In the case of hydrophilic matrices, greater than 20% of excipients were needed to control the amount of drug release within the first hours. With KMC, high level of a hydrophilic release retardant would be required to retard drug release because hydration/gel formation of hydrophilic polymers was hindered by the high levels of ions present in 10 meq of KMC following solubilization. This was undesirable because sustained release of approximately 10 mEq KMC tablets already contained 1.5 gram of active drug substance. The size of the tablet would be undesirably large for patients if a 20% drug release retardant were needed in the core tablet. In addition, a 3% subcoat and 6% enteric coating are needed to make the core tablet resistant to gastric fluid.

The inventors have found that solid oral dosage pharmaceutical preparations comprising polyvinylpyrrolidone at 1.0% (w/w) to 25% (w/w) and any cross-linked polyacrylic acid (CPAA), including, but not limited to those commercially available under the name “Carbopol®”, at 0.5% (w/w) to 10% (w/w) and preferably a wax component, and having a polyvinylpyrrolidone:Carbopol ratio of 1:5 to 5:1 has a superior short term, slow release profile for various active pharmaceutical ingredients, particularly for water soluble, alkali metal and/or alkaline earth metal salts which are provided in large doses (greater than or equal to 1 g/dosage unit).

### BRIEF SUMMARY OF THE INVENTION

The present invention relates to a solid oral dosage pharmaceutical preparation and a method of making same.

In one aspect of the present invention, there is a solid oral dosage pharmaceutical preparation comprising an active pharmaceutical ingredient and polyvinylpyrrolidone at 1.0% (w/w) to 25% (w/w); CPAA at 0.5% (w/w) to 10% (w/w); wherein the active pharmaceutical ingredient is selected from the group consisting of: a water soluble alkali metal salt, a water soluble alkaline earth metal salt, a water soluble mixed alkali metal and alkaline earth metal salt; and, any combination thereof; and, wherein the preparation has a weight ratio of polyvinylpyrrolidone:CPAA of 1:5 to 5:1. In preferred embodiments, the preparation further comprises one or more waxes. In preferred embodiments, the one or more waxes is present at 1% (w/w) to 30% (w/w). Preferably, the one or more waxes comprises a natural wax. The preferred natural wax is carnauba wax. When present, carnauba wax is preferably present at from 8% (w/w) to 16% (w/w). In some embodiments, the one or more waxes comprises glyceryl monostearate. Preferably, the active pharmaceutical ingredient is selected from the group consisting of magnesium citrate, potassium citrate, potassium magnesium citrate, potassium bicarbonate, and any combination thereof. In some embodiments, the active pharmaceutical ingredient comprises at least one diuretic. When present, the at least one diuretic may be selected from the group consisting of hydrochlorothiazide, chlorothiazide, furosemide, methazolamide, acetazolamide, chlorthalidone, benzthiazide, bendroflumethiazide, cyclothiazide, hydroflumethiazide, indapamide, methyclothiazide, metolazone, polythiazide, quinethazone, trichlormethiazide, and any combination thereof. Preferably, the solid oral dosage pharmaceutical preparation is a compressed tablet. Preferably, the tablet has a hardness of greater than 10 KFU. More preferably, the tablet has a hardness of greater than 15 KFU. Preferably, the CPAA is selected from the group consisting of Carbopol® 974P, Carbopol® 934, and any combination thereof. In some embodiments, the pharmaceutical preparation of claim 1, further comprises PEG. When present, the PEG is preferably selected from the group consisting of PEG 8000, PEG 6000, PEG 4000, and any combination thereof. Preferably, the polyvinylpyrrolidone is selected from the group consisting of Povidone K25, Povidone K30, Povidone K60, Povidone K90, and any combination thereof. In some embodiments, the CPAA is at a level of 0.5% (w/w) to 5% (w/w). In some embodiments, the preparation has a weight ratio of polyvinylpyrrolidone:CPAA of 1:1 to 5:1.

In another aspect of the present invention, there is a method of making a pharmaceutical solid oral solid oral dosage form comprising the steps of: forming a composition comprising an active pharmaceutical ingredient and polyvinylpyrrolidone at 1.0% (w/w) to 25% (w/w) and CPAA at 0.5% (w/w) to 15% (w/w), and having a polyvinylpyrrolidone:CPAA ratio of 1:5 to 5:1, wherein the active pharmaceutical ingredient is selected from the group consisting of: a water soluble alkali metal salt, a water soluble alkaline earth metal salt, a water soluble mixed alkali metal and alkaline earth metal salt; and, any combination thereof; and, compressing the composition into a solid oral dosage form. In some embodiments, the composition further comprises In some embodiments, the composition further comprises magnesium stearate. In preferred embodiments, the composition further comprises at least one wax. In some embodiments, the at least one wax comprises carnauba wax, glyceryl monostearate or a combination thereof. Preferably, the step of forming comprises mixing the polyvinylpyrrolidone, CPAA, and the wax and heating the mixture above the melting temperature of the wax. In some embodiments, the mixing is performed in a ribbon mixer. In some embodiments, the step of forming comprises forming a granulate by mixing and granulating the active pharmaceutical ingredient and the polyvinylpyrrolidone dissolved in a liquid medium to form a granulate, drying the granulate at a temperature above room temperature, and blending the granulation with CPAA. In preferred embodiments using a liquid medium, the liquid medium is an organic solvent. In preferred embodiments using a liquid medium of an organic solvent, the organic solvent is isopropyl alcohol. In some embodiments, the liquid medium is water. In some embodiments, the method further comprises the step of blending magnesium stearate after the step of blending the granulation with CPAA. In some embodiments, the step of granulating comprises granulating with a high speed/high shear granulator. In some embodiments, the step of granulating comprises granulating with a fluid bed granulator. In some embodiments comprising granulating with a fluid bed granulator, the CPAA is blended as a dry powder with dried active pharmaceutical ingredient after the active pharmaceutical ingredient was granulated with a solution of PVP. In some embodiments, the method further comprises the step of sieving the composition. In preferred embodiments, the step of compressing the composition into a solid oral dosage form comprises compressing the composition into a tablet. In preferred embodiments, the step of compressing the composition into a tablet comprises compressing the tablet to a hardness of greater than 10 KFU. In more preferred embodiments, the step of compressing the composition into a tablet comprises compressing the tablet to a hardness of greater than 15 KFU. In preferred embodiments, the active pharmaceutical ingredient is selected from the group consisting of magnesium citrate, potassium citrate, potassium magnesium citrate, potassium bicarbonate, and any combination thereof. In some embodiments, the preparation has a weight ratio of polyvinylpyrrolidone:CPAA of 1:1 to 5:1.

In another aspect of the present invention, there is a solid oral dosage pharmaceutical preparation comprising active pharmaceutical ingredient and polyvinylpyrrolidone at 1.0% (w/w) to 25% (w/w); CPAA at 0.5% (w/w) to 10% (w/w); and, wherein the preparation has a weight ratio of polyvinylpyrrolidone:CPAA of 1:5 to 5:1.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWING

For a more complete understanding of the present invention, reference is now made to the following descriptions taken in conjunction with the accompanying drawing, in which:

FIG. 1 shows the dissolution profile for 10 mEq potassium magnesium citrate tablets of Example 3 for potassium release.

FIG. 2 shows the dissolution profile for 10 mEq potassium magnesium citrate tablets of Example 3 for magnesium release.

### DETAILED DESCRIPTION OF THE INVENTION

As used herein, “a” or “an” means one or more. Unless otherwise indicated, the singular contains the plural and the plural contains the singular. For example, where reference is made to an active pharmaceutical ingredient, it should be understood that this encompasses at least one active pharmaceutical ingredient. Additionally, “a water soluble alkali metal salt” should be understood that this encompasses at least one water soluble alkali metal salt, etc.

In the present invention, a short term slow release drug delivery system comprising rapidly swelling and erodible polymeric substances, which upon contact with aqueous fluids, including gastric and intestinal fluids, and various adjuvants, slowly release active pharmaceutical ingredients.

The polymeric substances comprise polyvinylpyrrolidone (also known as “PVP” or “povidone” and is a polymer of 1-vinylpyrrolidone) and cross-linked polyacrylic acid. The amount of said polymeric substances in the formulation is from 0.5% (w/w) to 10% (w/w) of cross-linked polyacrylic acid and from 1.0% (w/w) to 25% (w/w) of PVP with relative ratios of PVP:cross-linked polyacrylic acid of 1:5 to 5:1, more preferably 1:1 to 5:1.

Herein, polyvinylpyrrolidone, PVP, and povidone are used synonymously and encompass any polymer of 1-vinylpyrrolidone and can be a homopolymer of I-vinylpyrrolidone or copolymer of 1-vinylpyrrolidone with one or more comonomers. Potential copolymers include for example, a copolymer of 1-vinylpyrrolidone and vinyl acetate.

Other excipients of typical pharmaceutical practice can be used as adjuvants, such as for example magnesium stearate or other metal stearates, stearic acid, colloidal silica, etc. Other additives such as glidants and plasticizers may be added to improve the addition of PVP on the surface of the active drug particles. Such additives may not only improve the manufacturing process, but also enhance the synergistic interaction between PVP and cross-linked polyacrylic acid. It should be understood that the excipients discussed are merely illustrative and do not represent an exhaustive list of possible excipients that can be used with the base composition.

The slow release formulation consists of rapidly swelling and/or gelable and/or erodible polymeric substances by contact with aqueous fluids and convenient adjuvants.

Compaction-enhancing agents are preferred in the preparation of the present invention. Compaction-enhancing agents are selected from the group consisting of hydrogenated castor oil, fatty acids, substituted triglycerides and glycerides, various grades of polyethylene glycols (PEG) and derivatives thereof having a different molecular weight generally ranging from 400 to 60,000. PEG is α-hydro-ω-hydroxy-poly(oxy-1,2-ethanediyl). PEG is preferred, with preferred grades being PEG 8000, PEG 6000, and PEG 4000. Other PEG grades are also useful in the present invention.

Other pharmaceutical inactive ingredients may also be used in the preparation of the present invention. Such ingredients are known to those of ordinary skill in the art.

Carbopol® (also known as Carbomer®) is a class of synthetic high molecular weight polymers of acrylic acid cross-linked with, for example, allyl ether of sucrose, allyl ether of pentaerythritol, and allyl ether of propylene. All grades of Carbopol® are useful in the present invention. Preferred grades of Carbopol® include Carbopol® 974P, Carbopol®1934, and any other Carbopol® approved for use in humans by official regulatory agencies. Where specific grades of Carbopol® are referenced, it should be understood that this also encompasses their equivalents. The cross-linked polyacrylic acid used herein is any cross-linked polyacrylic acid, particularly those approved for use in pharmaceutical preparations for humans by official regulatory agencies. In this way, the term “cross-linked polyacrylic acid” (also referred to herein as “CPAA”) as used herein refers to all of the commercial varieties of synthetic high molecular weight cross-linked polymers of acrylic acid (i.e., Carbopol®) as well as any other cross-linked polyacrylic acid.

All grades of PVP are useful in the present invention. Preferred grades of PVP include Povidone K25, Povidone K30, Povidone K60, and Povidone K90. Where specific grades of Carbopol® are referenced, it should be understood that this also encompasses their equivalents.

The PVP:CPAA compositions of the present represent a superior controlled release system which affords only slow ingress and egress of the active pharmaceutical ingredient.

Any solid oral dosage form may then be made with the pharmaceutical preparation of the present invention. Preferably, the solid oral dosage form is a compressed tablet. The compressed tablets made from the pharmaceutical preparation of the invention may be prepared from granular mixtures according to current production techniques. One non-limiting example is the preparation from ribbon mixer blends.

The inventors have found that superior short term slow release oral dosage drug delivery systems can be formulated which comprise PVP and CPAA in a range of PVP:CPAA of 1:5 to 5:1 (preferably a range of PVP:CPAA of 1:1 to 5:1) wherein the formulation comprises from 0.5% (w/w) to 10% (w/w) of CPAA and from 1.0% (w/w) to 25% (w/w) of PVP. Preferably, the formulation comprises from 0.5% (w/w) to 5% (w/w) of CPAA. It has also been found that the addition of a natural or synthetic wax provides structural and chemical benefits to the short term slow release systems. In embodiments having wax, the wax is preferably present at levels from 1% (w/w) to about 30% (w/w). The structural benefits accrue from the fact that the wax coated active ingredient in conjunction with the PVP and CPAA rate retarding components, do not disintegrate immediately upon ingestion, but rather erode slowly to assist in the slow release of active pharmaceutical ingredient. Additionally, by providing a hydrophobic environment for active pharmaceutical ingredients, the entry of water (which facilitates release of the active) into the matrix is retarded. The wax component aids the compaction properties of the formulation, resulting in a harder tablet which is more manufacturing-friendly.

In other embodiments, in addition to PVP, the formulation comprises from 0.5% (w/w) to 9% (w/w) of CPAA, from 0.5% (w/w) to 8% (w/w) of CPAA, from 0.5% (w/w) to 7% (w/w) of CPAA, from 0.5% (w/w) to 6% (w/w) of CPAA, with a preferable range of from 0.5% (w/w) to 5% (w/w) of CPAA. In other embodiments, the formulation comprises from 0.5% (w/w) to 4% (w/w) of CPAA, from 0.5% (w/w) to 3% (w/w) of CPAA, from 0.5% (w/w) to 2% (w/w) of CPAA, from 0.5% (w/w) to 1% (w/w) of CPAA, or from 0.5% (w/w) to less than 1% (w/w) of CPAA.

In other embodiments, in addition to CPAA, the formulation comprises from 1.0% (w/w) to 24% (w/w) of PVP, from 1.0% (w/w) to 23% (w/w) of PVP, from 1.0% (w/w) to 22% (w/w) of PVP, from 1.0% (w/w) to 21% (w/w) of PVP, from 1.0% (w/w) to 20% (w/w) of PVP, from 1.0% (w/w) to 19% (w/w) of PVP, from 1.0% (w/w) to 18% (w/w) of PVP, from 1.0% (w/w) to 17% (w/w) of PVP, from 1.0% (w/w) to 16% (w/w) of PVP, from 1.0% (w/w) to 15% (w/w) of PVP, from 1.0% (w/w) to 14% (w/w) of PVP, from 1.0% (w/w) to 13% (w/w) of PVP, from 1.0% (w/w) to 12% (w/w) of PVP, from 1.0% (w/w) to 11% (w/w) of PVP, or from 1.0% (w/w) to 10% (w/w) of PVP.

Several active pharmaceutical ingredients may be present in the formulation of the present invention. In preferred embodiments, the active pharmaceutical ingredient is one or more of any water soluble, alkaline salt, provided in large doses (greater than or equal to 1 g/dosage unit). As used herein, water soluble is defined as a minimum of 20 g/100 mL water (with a preferable range of 30-200 g/100 mL). Most preferably, the active pharmaceutical ingredient is one or more of any water soluble, alkaline salt, preferably an alkali metal salt, an alkaline earth metal salt, a mixed alkali metal and alkaline earth metal salt, preferably a potassium salt, provided in large doses (greater than or equal to 1 g/dosage unit). The active pharmaceutical ingredient may be any combination of the foregoing. The alkaline salts of potassium are each useful in the treatment and prevention of hypokalemia, thiazide induced hypokalemia as well as uric acid and calcium oxalate kidney stones. It is difficult to produce a suitable pharmaceutical product using these potassium salts because they are all relatively large molecules, all are somewhat hygroscopic and because the potassium ion is both irritating and somewhat erosive to the gastric mucosa, each must be given in a slow release form. Historically, wax matrix tablets or microencapsulated products have been used to avoid poor patient acceptance and poor dosage compliance. The present invention is particularly useful where dosage unit levels of the water soluble, alkaline salt are high; greater than 7 mEq and more preferably greater than 10 mEq.

Non-limiting examples of active pharmaceutical ingredients of alkali metal salts include potassium citrate and potassium bicarbonate. A non-limiting example of active pharmaceutical ingredients of an alkaline earth metal salt include magnesium citrate. A non-limiting example of active pharmaceutical ingredients of a mixed alkali metal and alkaline earth metal salt includes potassium magnesium citrate. Potassium magnesium citrate, when used in the present invention, can be of any stoichiometry, including 1:1:1 and 4:2:1, as well as other stoichiometries.

Optionally, where the formulation of the present invention may further comprise one or more diuretic active components. Exemplary diuretics include, but are not limited to, hydrochlorothiazide, chlorothiazide, furosemide, methazolamide, acetazolamide, chlorthalidone, benzthiazide, bendroflumethiazide, cyclothiazide, hydroflumethiazide, indapamide, methyclothiazide, metolazone, polythiazide, quinethazone, and trichlormethiazide. These are particularly preferred in combination with an active pharmaceutical ingredient of one or more alkaline salt, preferably an alkali metal salt or an alkaline earth metal salt, preferably a potassium salt.

It has proven very difficult to produce a well-tolerated high dosage content product. In general, patients are required to take multiple tablets, or multiple dosage regimens to accomplish good therapeutic or physiological steady state levels of potassium. The PVP:CPAA system disclosed herein provides a well-tolerated dosage with greater active dosage per tablet (upwards of 50% or more), using a very efficient combination of retardants requiring about one-half the total volume of retarding excipient materials normally needed to achieve a desirable release rate. Although the new pharmaceutical composition can be prepared by any process, below are provided some non-limiting examples of formulations and processes useful in the present invention. Two of the formulations have a wax component as a hydrophobic sealant and use ribbon mixer-based processes. The remaining two formulations do not include a wax component as a hydrophobic sealant and are granulator-based processes; one using a high shear/high speed granulator, and the other using a fluid bed granulator. These examples are not intended to be limiting as any process to produce a solid oral dosage pharmaceutical preparation comprising at least one active pharmaceutical ingredient and polyvinylpyrrolidone at 1.0% (w/w) to 25% (w/w); CPAA at 0.5% (w/w) to 5% (w/w); where the preparation has a weight ratio of polyvinylpyrrolidone:CPAA of 1:1 to 5:1 will suffice.

### EXAMPLE 1

### Wax Sealant Formulation in Ribbon Mixer, 2:1 PVP:CPAA

In one example of the drug release system of the present invention, there is a short term slow release drug delivery system having at least one water-soluble, alkali or alkaline earth metal salt for doses of greater than 1 g/dosage unit, the system comprising PVP and CPAA in a range of PVP:CPAA of 1:1 to 5:1 wherein the formulation comprises from 1.0% (w/w) to 25% (w/w) of PVP and from 0.5% (w/w) to 5% (w/w) of CPAA. In this embodiment, the system further comprises a wax as a hydrophobic sealant. In a preferred embodiment the wax is a natural wax, and the preferred natural wax is carnauba wax. Other natural waxes, known to be useful in pharmaceutical preparations by those of ordinary skill in the art are also applicable. Other example of natural waxes which can be used alternatively, or in combination, include beeswax, spermaceti, and paraffin wax. The list provided is merely illustrative and non-exhaustive of the possible natural waxes that can be used in the present invention.

An exemplary process description for this embodiment follows:
1. The active pharmaceutical ingredient (API), which is water soluble, hygroscopic, and alkaline in nature, is mixed with the primary binder (PVP), the release retardant (Carbopol®), and the hydrophobic sealant (carnauba wax).
2. The API, binder, and release retardants are coated with the sealant using a hot mix process in which the materials are heated to the melting point of the sealant to facilitate the coating process. A ribbon mixer with jacketed walls is used such that hot water (or other medium) can be used to heat the mixture above the melting temperature of the hydrophobic sealant (carnauba wax).
3. The product is cooled to ensure complete solidification.
4. The product is classified using a mill and sifter to achieve the particle size range that results in the desired release rate of the API in the finished tablet.
5. The classified material is blended with the secondary binder (PEG 8000) and tablet lubricant (magnesium stearate), and tableted.

Three different lots of tablets were manufactured with potassium citrate as the active pharmaceutical ingredient (15 mEq). The active pharmaceutical ingredient in the three lots of tablets is potassium citrate monohydrate. Although potassium citrate was used, it should be understood that one or more of any of the possible actives generally and specifically disclosed herein can be used. The same is true for all of the examples provided herein. The following table provides product formulas, tablet properties, product particle size distribution, and potassium dissolution data for the three tablet lots.

| | Lot # | | |
|---|---|---|---|
| | 1 | 2 | 3 |
| Product Formula | mg/T | mg/T | mg/T |
| Potassium Citrate Monohydrate | 1622.0 | 1622.0 | 1622.0 |
| Carnauba Wax | 202.2 | 202.2 | 202.2 |
| PVP K30 | 100.0 | 100.0 | 100.0 |
| CPAA 974P | 50.0 | 50.0 | 50.0 |
| Polyethylene Glycol 8000 | 50.0 | 50.0 | 50.0 |
| Magnesium Stearate | 1.0 | 1.0 | 1.0 |
| Total | 2025.2 | 2025.2 | 2025.2 |
| Product Formula | (% w/w) | (% w/w) | (% w/w) |
| Potassium Citrate Monohydrate | 80.09% | 80.09% | 80.09% |
| Carnauba Wax | 9.98% | 9.98% | 9.98% |
| PVP K30 | 4.94% | 4.94% | 4.94% |
| Carbopol 974P | 2.47% | 2.47% | 2.47% |
| Polyethylene Glycol 8000 | 2.47% | 2.47% | 2.47% |
| Magnesium Stearate | 0.05% | 0.05% | 0.05% |
| Total | 100.00% | 100.00% | 100.00% |
| Tablet Properties | | | |
| Target Tablet Weight (mg) | 2025.2 | 2025.2 | 2025.2 |
| Tablet Hardness (KFU) | 17.35 | 14.70 | 17.70 |
| Tablet Thickness (mm) | 8.50 | 8.58 | 8.47 |
| Tablet Friability, % | 1.16 | 1.88 | 1.20 |
| Product Particle Size Distribution | % | % | % |
| U.S. Standard Sieve Number | Retained | Retained | Retained |
| 14 | 0.01 | 0.06 | 0.08 |
| 20 | 0.72 | 0.42 | 0.33 |
| 30 | 6.41 | 2.96 | 3.69 |
| 40 | 29.65 | 25.23 | 27.50 |
| 60 | 44.46 | 47.60 | 46.86 |
| 80 | 9.72 | 12.7 | 12.05 |
| 100 | 2.54 | 3.67 | 3.45 |
| 120 | 1.6 | 2.43 | 1.88 |
| 140 | 1.57 | 2.35 | 1.73 |
| 170 | 1.09 | 0.84 | 0.75 |
| 200 | 1.01 | 1.00 | 0.83 |
| 325 | 1.21 | 0.74 | 0.80 |
| Pan | 0.13 | 0.09 | 0.09 |
| | % Label | % Label | % Label |
| Potassium Dissolution Data | | | |
| Potassium Dissolution (in H₂O) | | | |
| 0.5 Hour | 40.9 | 37.4 | 36.3 |
| 1 Hour | 64.6 | 57.5 | 56.3 |
| 3 Hour | 104.4 | 101.2 | 98.9 |
| Potassium Dissolution | | | |
| (in 0.1N HCl) | | | |
| 0.5 Hour | 35.4 | | |
| 1 Hour | 54.6 | | |
| 3 Hour | 103.4 | | |
| Potassium Dissolution | | | |
| (in Citrate Buffer) | | | |
| 0.5 Hour | 35.2 | | |
| 1 Hour | 56.8 | | |
| 3 Hour | 102.1 | | |

The same process was employed to manufacture 10 mEq tablets of potassium magnesium citrate. The following table provides product formulas, tablet properties, product particle size distributions, and potassium and magnesium dissolution data for this tablet lot.

| Product Formula | (mg/T) |
|---|---|
| Potassium Magnesium Citrate | 1470.3 |
| Carnauba Wax | 268.7 |
| Povidone K30 | 35.7 |
| Carbopol 974P | 17.9 |
| Polyethylene Glycol 8000 | 50.0 |
| Magnesium Stearate | 1.0 |
| Total | 1843.6 |
| Product Formula | (% w/w) |
| Potassium Magnesium Citrate | 79.75% |
| Carnauba Wax | 14.57% |
| Povidone K30 | 1.94% |
| Carbopol 974P | 0.97% |
| Polyethylene Glycol 8000 | 2.71% |
| Magnesium Stearate | 0.05% |
| Total | 100.00% |
| Tablet Properties | |
| Target Tablet Weight (mg) | 1843.6 |
| Tablet Hardness (KFU) | 21.55 |
| Tablet Thickness (mm) | 8.79 |
| Tablet Friability, % | 0.53 |
| Product Particle Size Distribution | % |
| U.S. Standard Sieve Number | Retained |
| 14 | 0.00 |
| 20 | 0.04 |
| 30 | 0.75 |
| 40 | 1.79 |
| 60 | 7.05 |
| 80 | 15.59 |
| 100 | 33.9 |
| 120 | 24.36 |
| 140 | 8.10 |
| 170 | 6.07 |
| 200 | 1.05 |
| 325 | 1.15 |
| Pan | 0.04 |
| | % Label |
| Potassium Dissolution Data | |
| Potassium Dissolution (Sodium Phosphate | |
| Buffer) | |
| 1 Hr | 35.9 |
| 2 Hr | 56.6 |
| 3 Hr | 77.6 |
| 4 Hr | 93.0 |
| 5 Hr | 95.6 |
| Magnesium Dissolution Data | |
| Magnesium Dissolution (Sodium Phosphate | |
| Buffer) | |
| 1 Hr | 36.6 |
| 2 Hr | 58.3 |
| 3 Hr | 81.4 |
| 4 Hr | 94.9 |
| 5 Hr | 101.9 |

In another embodiment, there is a system otherwise identical to that described above, but comprising a synthetic wax as a hydrophobic sealant. In a preferred embodiment the synthetic is glyceryl monostearate. Other example of synthetic waxes which can be used alternatively, or in combination, are erythritol distearate, glyceryl monostearate self-emulsifying, cetyl esters wax, and microcrystalline wax. The list provided is merely illustrative and non-exhaustive of the possible synthetic waxes that can be used in the present invention. The general process description for this embodiment is identical to that of the first example with the exception being the substitution of glyceryl monosterate for carnauba wax.

### EXAMPLE 2

### Synthetic and Natural Wax Sealant Formulation in Ribbon Mixer, Vary PVP:CPAA Ratio

Variations of the system of Example 1 were manufactured, studying the effects of substituting a synthetic wax (glyceryl monostearate) for the natural wax (carnauba wax) and varying the PVP:CPAA ratio. In this example, three lots of tablets were manufactured according to the process description of Example 1.

| | Lot # | | |
|---|---|---|---|
| | 1 | 2 | 3 |
| Product Formula | (mg/T) | (mg/T) | (mg/T) |
| Potassium Citrate Monohydrate | 1622.0 | 1622.0 | 1622.0 |
| Carnauba Wax | 202.2 | 202.2 | 0.0 |
| Glyceryl Monostearate | 0.0 | 0.0 | 245.0 |
| PVP K30 | 50.0 | 150.0 | 100.0 |
| Carbopol 974P | 50.0 | 50.0 | 50.0 |
| Polyethylene Glycol 8000 | 50.0 | 50.0 | 25.0 |
| Magnesium Stearate | 1.0 | 1.0 | 0.5 |
| Total | 1975.2 | 2075.2 | 2042.5 |
| PVP:CPAA Ratio | 1:1 | 3:1 | 2:1 |
| Product Formula | (% w/w) | (% w/w) | (% w/w) |
| Potassium Citrate Monohydrate | 82.12% | 78.16% | 79.41% |
| Carnauba Wax | 10.24% | 9.74% | 0.00% |
| Glyceryl Monostearate | 0.00% | 0.00% | 12.00% |
| PVP K30 | 2.53% | 7.23% | 4.90% |
| Carbopol 974P | 2.53% | 2.41% | 2.45% |
| Polyethylene Glycol 8000 | 2.53% | 2.41% | 1.22% |
| Magnesium Stearate | 0.05% | 0.05% | 0.02% |
| Total | 100.00% | 100.00% | 100.00% |
| Tablet Properties | | | |
| Target Tablet Weight (mg) | 1975.2 | 2075.2 | 2042.5 |
| Tablet Hardness (KFU) | 14.40 | 12.26 | 14.44 |
| Tablet Thickness (mm) | 8.48 | 9.00 | 8.75 |
| Tablet Friability, % | 1.26 | 1.14 | 0.16 |
| Product Particle Size Distribution | % | % | % |
| U.S. Standard Sieve Number | Retained | Retained | Retained |
| 14 | 0.08 | 0.06 | 0.06 |
| 20 | 0.27 | 0.25 | 1.05 |
| 30 | 6.18 | 4.02 | 9.68 |
| 40 | 32.62 | 32.00 | 28.98 |
| 60 | 46.30 | 45.24 | 32.28 |
| 80 | 8.80 | 9.45 | 9.67 |
| 100 | 2.27 | 3.50 | 6.07 |
| 120 | 1.27 | 1.63 | 3.26 |
| 140 | 0.77 | 1.41 | 1.65 |
| 170 | 0.80 | 1.82 | 4.91 |
| 200 | 0.42 | 0.43 | 1.32 |
| 325 | 0.26 | 0.33 | 1.24 |
| Pan | 0.00 | 0.00 | 0.01 |
| Potassium Dissolution Data | | | |
| Potassium Dissolution (in H₂O) | % Label | % Label | % Label |
| 0.5 Hour | 43.9 | 39.2 | 29.8 |
| 1 Hour | 70.8 | 60.7 | 38.9 |
| 3 Hour | 107.3 | 100.1 | 70.3 |

As can be seen variation of the PVP:CPAA ratio can be used to modify the release profile of the drug delivery system. Similarly, use of different hydrophobic sealants can also be used to modify the release profile of the drug delivery system. Glyceryl monostearate is an octadecanoic acid, monoester with 1,2,3,-propane-triol. Preferred tablet hardness is greater than 10 KFU (kilopond force unit), more preferably greater than 15 KFU.

### EXAMPLE 3

### PVP/Isopropyl Alcohol Solution in High Speed/High Shear Granulators

In another example of the drug release system of the present invention, there is a short term slow release drug delivery system having at least one water-soluble, alkali or alkaline earth metal salt for doses of greater than 1 g/dosage unit, the system comprising PVP and CPAA in a range of PVP:CPAA of 1:1 to 5:1 wherein the formulation comprises from 0.5% (w/w) to 5% (w/w) of CPAA and from 1.0% (w/w) to 25% (w/w) of PVP.

Linear drug release was achieved with the combination of PVP and CPAA as drug release retardants at a 9% level. Linear release characteristic assured both the absence of initial burst effect and the complete release at the later stage of the dissolution process. It was hypothesized there were synergistic effects between KMC, PVP and CPAA. KMC release rate was the slowest when PVP and CPAA were present at approximately a 1:1 ratio. PVP plus CPAA are present at a 9% level and at a ratio of 2:1 in the current lead formulation. This will allow both the desired release profile and good compaction properties of the final granules.

The alkaline nature of KMC induced faster gelation of CPAA that in turn retarded the release of KMC. Although not wishing to be bound by theory, it is believed that PVP forms hydrogen bonds with CPAA in the current drug delivery system. The extent of hydrogen bonding is enhanced in the presence of potassium and magnesium ions. When PVP was replaced with ethyl cellulose, linear drug release was absent.

In one embodiment, PVP was introduced to the formulation as a solution in isopropyl alcohol (IPA). However, other liquid media can be substituted for IPA. These include both other organic solvents and water. Use of IPA resulted in the formation of a thin layer of PVP film on the surface of the KMC particles. The thin layer of PVP on the surface of KMC granules improved the compaction properties of KMC significantly.

The mixing order of retardants was found to have a significant impact on the dissolution stability of KMC core tablet. Stable tablets were only achieved when CPAA was blended in as a dry powder following the drying of KMC that was previously granulated with PVP/IPA solution. It was hypothesized that the exposure of CPAA to the granulation medium, previous to drying, caused poor stability in drug release during storage.

An exemplary process description for this embodiment follows:
1. High shear granulate KMC with 12% PVP K30 solution in IPA as granulation medium.
2. Oven dry at 40° C. until LOD≦2%.
3. Blend granulation with Carbopol® 974P in a plastic bag for approximately 3 minutes.
4. Sieve (20 mesh) blend, blend again, and then sieve again.
5. Blend in Mg Stearate in a plastic bag (blending time was not recorded).
6. Compress the final blend into tablets (tablet hardness is approximately 30 Kp).
7. Apply Klucel EF in IPA at 3% coating level.
8. Apply Eudragit L30 D-55 at 6% coating level.

One lot of tablets were manufactured with potassium magnesium citrate (KMC) as the active pharmaceutical ingredient. Although KMC was used, it should be understood that one or more of any of the possible actives generally and specifically disclosed herein can be used. The following table provides product formulas, tablets properties, product particle size distribution, and potassium dissolution data for the manufactured lot.

| Product Formula | (mg/T) |
|---|---|
| Potassium Magnesium Citrate | 1500.0 |
| Povidone K30 | 100.0 |
| Carbopol 974P | 50.0 |
| Magnesium Stearate | 16.7 |
| Total | 1666.7 |
| Product Formula | (% w/w) |
| Potassium Magnesium Citrate | 90.00% |
| Povidone K30 | 6.00% |
| Carbopol 974P | 3.00% |
| Magnesium Stearate | 1.00% |
| Total | 100.00% |
| Tablet Properties | |
| Target Tablet Weight (mg) | 1666.7 |
| Tablet Hardness (KFU) | 23.55 |
| Tablet Thickness (mm) | 8.03 |
| Tablet Friability, % | 0.16 |
| Product Particle Size Distribution | |
| U.S. Standard Sieve Number | % Retained |
| 14 | not determined |
| 20 | not determined |
| 30 | 0.3 |
| 40 | 2.2 |
| 60 | 18.5 |
| 80 | 25.6 |
| 100 | not determined |
| 120 | 31.9 |
| 140 | 7.3 |
| 170 | not determined |
| 200 | 11.5 |
| 325 | not determined |
| Pan | 2.6 |

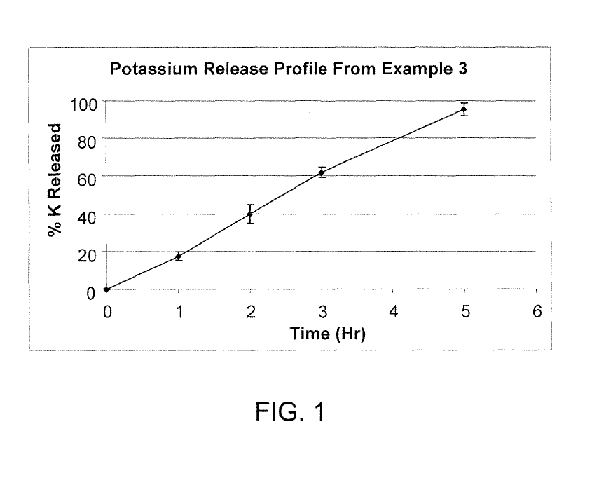

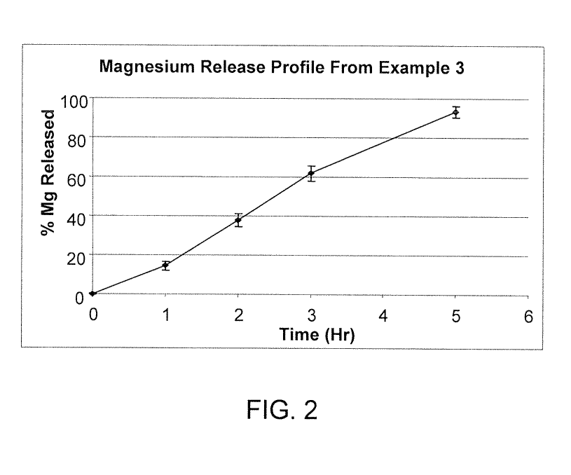

The dissolution profiles for this lot of tablets is provided in FIG. 1 for potassium release and in FIG. 2 for magnesium release.

### EXAMPLE 4

### PVP and CPAA Fluid Bed Granulators

Many techniques are available to produce solid dosage forms for pharmaceuticals. Pharmaceuticals are combined with excipients such as binders, preservatives, flavors, fillers, and coatings to produce the most effective formulation. Fluid bed dryers are used extensively in the pharmaceutical industry to coat active ingredients with binders using a solution containing the excipient sprayed into a mixture of air and the product. Different product forms result from the choice of operating parameters. For example, grape-like clusters of product may be formed in which the majority of the binder is present in the crevices attaching individual particles. Particles may also be coated individually if desired, sometimes with many different coating layers.

Two techniques were investigated for coating potassium magnesium citrate particles with a moderate molecular weight PVP (BASF Kollidon 30) in the two-liter bowl of a Fluid Air laboratory-scale fluid-bed dryer. In both techniques, the bed temperature was high throughout the spray process (38° C.), and the air-flow rate was moderate (30 scfm/kg). The coating flow rate was maintained at a low level (11 g/min-kg). These conditions were chosen to minimize the formation of agglomerates.

In the first technique, a top-spray approach using a granulation wand was used. A 10% aqueous solution of the PVP pumped through the wand and atomized with 18 psig air at a point about 4-5 inches above the fluidized bed until the product contained a 6% loading of PVP. The final product contained approximately 10% coarse product (>25 mesh) that was sieved out and discarded. The quantity in this coarse fraction was variable from batch to batch. The final product exhibited a great increase in particle size, and was quite agglomerated when viewed by environmental scanning electron microscopy (ESEM). A low degree of coating was observed on individual particles.

The second technique used a nozzle spraying from the bottom of the bed up through a cylindrical annulus though which the product was re-circulated. This is a traditional air suspension coating process developed by Dr. Dale Wurster in the 1950's and 1960's. In this process, the density of the flow into which the spray is injected can be adjusted by the gap between the annulus and the bottom of the chamber. This process more efficiently coats particles in a reproducible fashion, but is less easily implemented and more costly than top-spraying. Again, a 10% aqueous PVP solution was used to produce a final product that was loaded at 6% PVP. The final product was free flowing, and contained none of the agglomerates that were formed from the top-spray. Under ESEM, the particles exhibited agglomeration, but not the same degree as the particles from the top-spray experiments. The particles were coated to a greater degree, and the particle size was lower.

In another example of the drug release system of the present invention, there is a short term slow release drug delivery system having at least one water-soluble, alkali or alkaline earth metal salt for doses of greater than 1 g/dosage unit, the system comprising PVP and CPAA in a range of PVP:CPAA of 1:1 to 5:1 wherein the formulation comprises from 0.5% (w/w) to 5% (w/w) of CPAA and from 1.0% (w/w) to 25% (w/w) of PVP.

An exemplary process description for this embodiment follows:
1. Add 750.0 g of KMC to 3 liter bowl.
2. Dissolve 45.0 g of PVP (PVP K30/BASF) into 405 mL of DI water.
3. Preheat fluid bed to 55° C.
4. Begin flow of solution to granulating wand positioned 5-6 inches above fluid bed.
5. Use following Fluid Bed parameters:
   Inlet Temp: 80° C.
   Inlet Flow: 15 CFM
   Granulation Wand Used, 18 psi atomizing air with 5.5 mL/min solution flow
   Bed Temperature: Maintained at 40° C. with above solution flow
6. Collect samples at 1.5%, 3.0%, 4.5%, and 6.0% PVP coating levels.
7. Final weight was 734.4 g.
8. Sieve material through 25 mesh screen, and collected 88.3 g of reject and 646.1 g of product.
9. Add 3% by weight CPAA (20.2 g) to product and sieved to mix.
10. Place in fluid bed with 15 cfm flow for 4 min.
11. Add 1% by weight Mg Stearate (6.73 g) to bowl, and mixed at 15 cfm for an additional 1 min.

One lot of tablets were manufactured with potassium magnesium citrate (KMC) as the active pharmaceutical ingredient. Although KMC was used, it should be understood that one or more of any of the possible actives generally and specifically disclosed herein can be used. The following table provides product formulas, tablets properties, product particle size distribution, and potassium dissolution data for the manufactured lot.

| Product Formula | mg/T |
|---|---|
| Potassium Magnesium Citrate | 1500.0 |
| PVP K30 | 100.0 |
| Carbopol 974P | 50.0 |
| Magnesium Stearate | 16.7 |
| Total | 1666.7 |
| Product Formula | (% w/w) |
| Product Formula (% w/w) | % w/w |
| Potassium Magnesium Citrate | 90.00% |
| PVP K30 | 6.00% |
| Carbopol 974P | 3.00% |
| Magnesium Stearate | 1.00% |
| Total | 100.00% |
| Tablet Properties | |
| Target Tablet Weight (mg) | 1666.7 |
| Tablet Hardness (KFU) | 36.1 |
| Tablet Thickness (mm) | 8.03 |
| Tablet Friability, % | 0.13 |
| | % Label |
| Potassium Dissolution Data | |
| Potassium Dissolution (Sodium Phosphate Buffer) | |
| 1 Hour | 27.5 |
| 2 Hour | 49.4 |
| 3 Hour | 67.8 |
| 4 Hour | 83.5 |
| 5 Hour | 92.2 |
| Magnesium Dissolution Data | |
| Magnesium Dissolution (Sodium Phosphate Buffer) | |
| 1 Hour | 25.7 |
| 2 Hour | 54.2 |
| 3 Hour | 74.3 |
| 4 Hour | 95.2 |
| 5 Hour | 103.1 |

A nearly identical process to that described above in Example 4 was performed; the only significant change affecting in step 4 of the process above. In the modified process, the granulating wand is replaced with a Wurster Coating nozzle positioned below the fluid bed. Solution is thus added from below instead of from above the fluid bed. An example of the generalized process description for this modified process is provided below:
1. 500 g of KMC is coated with 30 g of Kollidon K-30 PVP within the two liter bowl of a Fluid-Air Fluid-Bed dryer equipped with a Wurster Coater.
2. Add 500 g of oven converted KMC to 2 L fluid bed bowl. Begin operation of fluid bed with following parameters:
3. Inlet temp: 80° C.
4. Air flow: 15 scfm.
5. Atomizing air at 18 psig.
6. Bed heated to about 55° C., and liquid flow was started at 5.5 g/min
7. Bed temperature equilibrated to about 40° C.
8. After addition of solution, an increase temperature to 50° C. indicates dryness.
9. Collect 511 g of product.
10. The process is repeated on 500 g of additional KMC and 514 g of product is collected.
11. Products are combined, and run through a 25 mesh screen with 32.03 g of CPAA.
12. The mixture is placed in fluid bed, and 10.68 g of magnesium stearate is added to top of the mixture.
13. The fluid bed dryer is turned on at 15 cfm for 10 minutes to mix the product.
14. A total of 1048 g of product is collected.

Another lot of tablets were manufactured with KMC as the active pharmaceutical ingredient. Although KMC was used, it should be understood that one or more of any of the possible actives generally and specifically disclosed herein can be used. The following table provides product formulas, tablets properties, product particle size distribution, and potassium dissolution data for the manufactured lot.

| Product Formula | mg/T |
|---|---|
| Potassium Magnesium Citrate | 1500.0 |
| Povidone K30 | 100.0 |
| Carbopol 974P | 50.0 |
| Magnesium Stearate | 16.7 |
| Total | 1666.7 |
| Product Formula (% w/w) | |
| Product Formula (% w/w) | % w/w |
| Potassium Magnesium Citrate | 90.00% |
| Povidone K30 | 6.00% |
| Carbopol 974P | 3.00% |
| Magnesium Stearate | 1.00% |
| Total | 100.00% |
| Tablet Properties | |
| Target Tablet Weight (mg) | 1666.7 |
| Tablet Hardness (KFU) | 34.29 |
| Tablet Thickness (mm) | 8.01 |
| | % Label |
| Potassium Dissolution Data | |
| Potassium Dissolution (Sodium Phosphate | |
| Buffer) | |
| 1 Hr | 23.5 |
| 2 Hr | 41.5 |
| 3 Hr | 59.6 |
| 4 Hr | 75.2 |
| 5 Hr | 86.8 |
| Magnesium Dissolution Data | |
| Magnesium Dissolution (Sodium Phosphate | |
| Buffer) | |
| 1 Hr | 19.1 |
| 2 Hr | 39.9 |
| 3 Hr | 57.6 |
| 4 Hr | 77.6 |
| 5 Hr | 93.7 |

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the composition of matter, and methods described in the specification. As one of ordinary skill in the art will readily appreciate from the disclosure of the present invention, compositions of matter, methods, or steps, presently existing or later to be developed that perform substantially the same function or achieve substantially the same result as the corresponding embodiments described herein may be utilized according to the present invention. Accordingly, the appended claims are intended to include within their scope such processes, compositions of matter, methods, or steps.

1. A solid oral dosage pharmaceutical preparation comprising an active pharmaceutical ingredient and
polyvinylpyrrolidone at 1.0% (w/w) to 25% (w/w);
CPAA at 0.5% (w/w) to 10% (w/w);
wherein said active pharmaceutical ingredient is selected from the group consisting of:
a water soluble alkali metal salt,
a water soluble alkaline earth metal salt,
a water soluble mixed alkali metal and alkaline earth metal salt; and,
any combination thereof
and,
wherein said preparation has a weight ratio of polyvinylpyrrolidone:CPAA of 1:5to 5:1.

2. The solid oral dosage pharmaceutical preparation of claim 1, further comprising at least one wax.

3. The solid oral dosage pharmaceutical preparation of claim 2, wherein said at least one wax is present at 1% (w/w) to 30% (w/w).

4. The solid oral dosage pharmaceutical preparation of claim 2, wherein said at least one wax comprises a natural wax.

5. The solid oral dosage pharmaceutical preparation of claim 4, wherein said natural wax is carnauba wax.

6. The solid oral dosage pharmaceutical preparation of claim 5, wherein said carnauba wax is present at from 8% (w/w) to 16% (w/w).

7. The solid oral dosage pharmaceutical preparation of claim 2, wherein said at least one wax comprises glyceryl monostearate.

8. The solid oral dosage pharmaceutical preparation of claim 1, wherein said active pharmaceutical ingredient is selected from the group consisting of magnesium citrate, potassium citrate, potassium magnesium citrate, potassium bicarbonate, and any combination thereof.

9. The solid oral dosage pharmaceutical preparation of claim 1, wherein said active pharmaceutical ingredient comprises at least one diuretic.

10. The solid oral dosage pharmaceutical preparation of claim 9, wherein said at least one diuretic is selected from the group consisting of hydrochlorothiazide, chlorothiazide, furosemide, methazolamide, acetazolamide, chlorthalidone, benzthiazide, bendroflumethiazide, cyclothiazide, hydroflumethiazide, indapamide, methyclothiazide, metolazone, polythiazide, quinethazone, trichlormethiazide, and any combination thereof.

11. The solid oral dosage pharmaceutical preparation of claim 1, wherein said solid oral dosage pharmaceutical preparation is a compressed tablet.

12. The solid oral dosage pharmaceutical preparation of claim 11, wherein said tablet has a hardness of greater than 10 KFU.

13. The solid oral dosage pharmaceutical preparation of claim 12, wherein said tablet has a hardness of greater than 15 KFU.

14. The solid oral dosage pharmaceutical preparation of claim 1, wherein said CPAA is selected from the group consisting of Carbopol® 974P, Carbopol® 934, and any combination thereof.

15. The solid oral dosage pharmaceutical preparation of claim 1, further comprising PEG.

16. The solid oral dosage pharmaceutical preparation of claim 15, wherein said PEG is selected from the group consisting of PEG 8000, PEG 6000, PEG 4000, and any combination thereof.

17. The solid oral dosage pharmaceutical preparation of claim 1, wherein said polyvinylpyrrolidone is selected from the group consisting of Povidone K25, Povidone K30, Povidone K60, Povidone K90, and any combination thereof.

18. The solid oral dosage pharmaceutical preparation of claim 1, wherein said CPAA is at a level of 0.5% (w/w) to 5% (w/w).

19. The solid oral dosage pharmaceutical preparation of claim 1, wherein said preparation has a weight ratio of polyvinylpyrrolidone:CPAA of 1:1 to 5:1.

20. A method of making a pharmaceutical solid oral dosage form comprising the steps of:
forming a composition comprising (a) an active pharmaceutical ingredient (b) polyvinylpyrrolidone at 1.0% (w/w) to 25% (w/w) and (c) CPAA at 0.5% (w/w) to 15% (w/w), and having a polyvinylpyrrolidone:CPAA ratio of 1:5to 5:1, wherein said active pharmaceutical ingredient is selected from the group consisting of:
a water soluble alkali metal salt,
a water soluble alkaline earth metal salt,
a water soluble mixed alkali metal and alkaline earth metal salt; and,
any combination thereof
and,
compressing said composition into a solid oral dosage form.

21. The method of claim 20, wherein said composition further comprises PEG.

22. The method of claim 20, wherein said composition further comprises magnesium stearate.

23. The method of claim 20, wherein said composition further comprises at least one wax.

24. The method of claim 23, wherein said at least one wax is selected from the group consisting of carnauba wax, glyceryl monostearate and any combination thereof.

25. The method of claim 24, wherein said step of forming comprises mixing said polyvinylpyrrolidone, CPAA, and said wax and heating said mixture above the melting temperature of said wax.

26. The method of claim 25, wherein said mixing is performed in a ribbon mixer.

27. The method of claim 20, wherein said step of forming comprises forming a granulate by mixing and granulating said active pharmaceutical ingredient and said polyvinylpyrrolidone dissolved in a liquid medium to form a granulate, drying said granulate at a temperature above room temperature, and blending said granulation with CPAA.

28. The method of claim 27, wherein said liquid medium is an organic solvent.

29. The method of claim 28, wherein said organic solvent is isopropyl alcohol.

30. The method of claim 27, wherein said liquid medium is water.

31. The method of claim 27, further comprising the step of blending magnesium stearate after said step of blending said granulation with CPAA.

32. The method of claim 27, wherein said step of granulating comprises granulating with a high speed/high shear granulator.

33. The method of claim 27, wherein said step of granulating comprises granulating with a fluid bed granulator.

34. The method of claim 33, wherein said CPAA is blended as a dry powder with dried active pharmaceutical ingredient after said active pharmaceutical ingredient is granulated with a solution of PVP.

35. The method of claim 20, further comprising the step of sieving said composition.

36. The method of claim 20, wherein said step of compressing said composition into a solid oral dosage form comprises compressing said composition into a tablet.

37. The method of claim 36, wherein said step of compressing said composition into a tablet comprises compressing said tablet to a hardness of greater than 10 KFU.

38. The method of claim 37, wherein said step of compressing said composition into a tablet comprises compressing said tablet to a hardness of greater than 15 KFU.

39. The method of claim 20, wherein said active pharmaceutical ingredient is selected from the group consisting of magnesium citrate, potassium citrate, potassium magnesium citrate, potassium bicarbonate, and any combination thereof.

40. The method of claim 20, wherein said preparation has a weight ratio of polyvinylpyrrolidone:CPAA of 1:1 to 5:1.

41. A solid oral dosage pharmaceutical preparation comprising active pharmaceutical ingredient and
polyvinylpyrrolidone at 1.0% (w/w) to 25% (w/w);
CPAA at 0.5% (w/w) to 10% (w/w);
and
wherein said preparation has a weight ratio of polyvinylpyrrolidone:CPAA of 1:5 to 5:1.
